(19) United States
(12) Patent Application Publication
(10) Pub. No.: US 2023/0128233 A1
(43) Pub. Date: Apr. 27, 2023

(54) TREATING MORBID OBESITY EXTRACORPOREALLY

(71) Applicant: Halberd Corporation, Jackson Center, PA (US)

(72) Inventor: Mitchell Steven Felder, Hermitage, PA (US)

(21) Appl. No.: 17/971,172

(22) Filed: Oct. 21, 2022

Related U.S. Application Data
(60) Provisional application No. 63/270,387, filed on Oct. 21, 2021.

(51) Int. Cl. A61M 1/36; C07K 16/18

(52) U.S. Cl. CPC A61M 1/3683; A61M 1/362; C07K 16/18; C07K 2319/60

(57) ABSTRACT

An embodiment provides a method for treating a body fluid of a patient with morbid obesity, including: removing the body fluid from a patient; applying a treatment to the body fluid, wherein the treatment comprises an antibody that joins with a morbid obesity targeted antigen (TA) in the body fluid to form an antibody-TA complex, wherein the antibody comprises a tag sensitive to an illumination; removing the antibody-TA complex from the body fluid using an illumination source; and returning the body fluid to the patient. Other aspects are described and claimed.

### CROSS REFERENCE TO RELATED APPLICATION

This application claims priority to U.S. Provisional Pat. Application Serial No. 63/270,387, filed on Oct. 21, 2021, and entitled “TREATING MORBID OBESITY EXTRACORPOREALLY,” the contents of which are incorporated by reference herein.

### FIELD

This application relates generally to a treatment for morbid obesity, and, more particularly, to an extracorporeal and laser methodology for the treatment of morbid obesity.

### BACKGROUND

Morbid obesity has been reported in all countries, where it has caused a great deal of death, suffering, and increased healthcare cost. Among adults under the age of seventy, obesity is the second leading cause of death each year. Tobacco use is the leading cause of death in these patients. Obesity is a major factor in the underlying cause of a large number of health conditions. These include diabetes, heart disease, stroke, hypertension, asthma, sleep apnea, gallstones, kidney stones, infertility, and as many as eleven types of cancers, including leukemia, breast, and colon cancer. No less real are the social and emotional effects of obesity, including discrimination, lower wages, lower quality of life and a likely susceptibility to depression.

### BRIEF SUMMARY

In summary, one embodiment provides a method for treating a body fluid of a patient with morbid obesity, comprising: removing the body fluid from a patient; applying a treatment to the body fluid, wherein the treatment comprises an antibody that joins with a morbid obesity targeted antigen (TA) in the body fluid to form an antibody-TA complex, wherein the antibody comprises a tag sensitive to an illumination; removing the antibody-TA complex from the body fluid using an illumination source; and returning the body fluid to the patient.

Another embodiment provides a method for treating a body fluid of a patient with morbid obesity, comprising: removing the body fluid from a patient; applying a treatment to the body fluid, wherein the treatment comprises an antibody that joins with a morbid obesity targeted antigen (TA) in the body fluid to form an antibody-TA complex, wherein the antibody comprises a tag sensitive to illumination and a metallic moiety sensitive to a radiofrequency; removing the antibody-TA complex from the body fluid using an illumination source; and returning the body fluid to the patient.

A further embodiment provides a device for treating a body fluid of a patient with morbid obesity, comprising: a transparent first stage including an inlet for the body fluid and at least one exterior wall defining a treatment chamber; a transparent second stage, fluidly connected to the first stage, comprising a removal module and an outlet for the body fluid, wherein the treatment chamber comprises a delivery tube for introducing an antibody into the treatment chamber, wherein the delivery tube comprises a hollow tube including at least one interior wall defining a plurality of holes through which the antibody can be added to the treatment chamber, wherein the treatment is delivered through the hollow tube in counter-current mode with reference to the body fluid, wherein the treatment comprises an antibody that joins with an Morbid obesity targeted antigen (TA) in the body fluid to form an antibody-TA complex, wherein the antibody comprises a tag sensitive to an illumination; and a laser illumination source.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example partial cross-sectional view of a cylinder and tubing used to deliver a treatment to a bodily fluid.

FIG. 2 illustrates an example a partial cross-sectional view showing additional detail of the cylinder and tubing of FIG. 1.

FIG. 3 illustrates an example series of microscreens for filtering a body fluid.

FIG. 4 illustrates an example flow diagram of a method for treatment of morbid obesity using a laser.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

There is therefore a need for treatments to reduce morbid obesity symptomatology in a clinical setting. Obesity is a major cause of morbidity, disability, and premature death. Obesity remains a major factor in the underlying cause of a large number of health conditions. These include diabetes, heart disease, stroke, hypertension, asthma, sleep apnea, gallstones, kidney stones, infertility, and as many as eleven types of cancers, including leukemia, breast, and colon cancer. Obesity may also reduce positive outcomes from other disease states or even sickness. Body mass index (BMI) remains a strong predictor of mortality among adults. Morbid obesity may be defined as a BMI of 40 or above. Morbid obesity may be defined at a BMI lower than 40 with other medical conditions such as diabetes, high blood pressure, or the like. Morbid obesity may be defined as an individual with a body weight 100 pounds greater than an ideal weight, for example based upon factors such as height, age, gender, body build type, waist size, body fat percentage, a ratio of body measurements, or the like. Morbid obesity may be defined by other medical standards and/or adjusted based upon an individual’s medical condition. However, the methodology described herein may be used for treatment of a patient with other levels of obesity. Morbid obesity may reduce life expectancy by a decade or more.

Accordingly, a method for the extracorporeal treatment of a patient’s body fluid, such as blood, with the utilization of laser technology and/or radiofrequency for the emissive energy eradication of the morbid obesity Target Antigens (TAs): IL-1 (Interleukin-1), IL-6 (Interleukin-6), TNF-alpha (Tumor Necrosis Factor-alpha), cholesterol, C-Reactive Protein (CRP), Alpha-2-HS-glycoprotein (Fetuin-A), Inhibitor of Differentiation 1 (ID1), protein-tyrosine phosphatase (PTP)1B, monocyte chemoattractant protein-1 (MCP-1), IL-8 (Interleukin-8), haptoglobin, α-1 antitrypsin, α-1 acid-glycoprotein, and/or orosomucoid in the body fluid, blood or blood plasma, to form an antibody-TA complex, wherein the antibody comprises a tag sensitive to an illumination; in said body fluid is described herein. The treatment includes stages comprising removing the body fluid from a patient diagnosed with morbid obesity, exposing and binding one or more combinations of those morbid obesity Target Antigen(s) to fluorescent or luminous antibodies forming antibody complexes, eradicating the fluorescently or luminously antibody complexes with a laser, and then returning the body fluid to the patient.

The illustrated example embodiments will be best understood by reference to the figures. The following description is intended only by way of example, and simply illustrates certain example embodiments.

In an embodiment, the method may comprise treating a patient’s body fluid (blood plasma and/or blood) extracorporeally with fluorescently or luminously conjugated antibody(s). The antibody(s) may be designed to react with and bind up particular targeted antigen(s)/TA(s) of morbid obesity that include, but are not limited to: IL-1 (Interleukin-1), IL-6 (Interleukin-6), TNF-alpha (Tumor Necrosis Factor-alpha), cholesterol, C-Reactive Protein (CRP), Alpha-2-HS-glycoprotein (Fetuin-A), Inhibitor of Differentiation 1 (ID1), protein-tyrosine phosphatase (PTP)1B, monocyte chemoattractant protein-1 (MCP-1), IL-8 (Interleukin-8), haptoglobin, α-1 antitrypsin, α-1 acid-glycoprotein, and/or orosomucoid in the body fluid to form an antibody-TA complex, wherein the antibody comprises a tag sensitive to an illumination.

In an embodiment, a short-duration pulse-beam from a laser or other high energy radiation emissive source may then be used to eradicate the morbid obesity Target Antigen(s) -antibody complexes in the body fluid (blood plasma and/or blood), and then the body fluid may be returned back to the patient.

In an embodiment, in the first stage, a body fluid (e.g., blood plasma or blood) may be withdrawn from a patient using standard medical techniques. One convenient method for removing blood may be the standard venipuncture technique. Other techniques known to those skilled in the art are contemplated by this disclosure.

In an embodiment, in the second stage a treatment may be applied to a body fluid extracorporeally. The treatment may comprise exposing the body fluid to a fluorescent or luminous tagged antibody (F/LT Ab) generated to bind specific targeted pathogenic antigens (TPAs) or targeted anitgens (TAs) of morbid obesity such as those described above. During this treatment the fluorescently tagged or luminously conjugated antibody(s) and the targeted pathogen antigen form morbid obesity fluorescent antibody complexes (F/LT Ab-TA complexes). Using an extremely narrow beam laser as an eradication tool, these morbid obesity antibody complexes may be substantially eliminated from the extracorporeal body fluid. In an embodiment, the laser beam may be less than 10 nanometers in diameter.

In an embodiment, one method of eradication may include using an illumination system comprising an optic or other suitable sensor for detecting individual F/LT Ab-TA complexes in the extracorporeal body fluid. Additionally, in an embodiment, a high energy radiation source, such as a very narrow beam laser (for example, less than 10 nm in diameter), or another coherent light beam for eradicating the antibody complexes may be used. The body fluid may be pumped past the sensor where the body fluid may be illuminated by various techniques known in the art and the F/LT Ab-TAs can be identified by various techniques known in the art.

In an embodiment, a non-limiting generalized example is as follows: the sensor may be connected to a control unit. The signal from the sensed F/LT Ab-TA complexes may be transmitted to a control unit which controls a high energy emissive source. The receipt of a F/LT Ab-TA signal causes the control unit to emit a short-duration pulse-beam from a laser or other high energy radiation emissive source. The energy of the emitted radiation annihilates the F/LT Ab-TA, thereby destroying its disease-causing potential. The entire system may be monitored and controlled utilizing a computer, in real time, utilizing time units of 1 millisecond or less during the entire procedure. Persons having ordinary skill in art will recognize that the steps described above can be performed on various devices, machines, or systems. This disclosure contemplates all known devices, machines, or systems that can perform the steps described in the above illustrative example.

In an embodiment, the second stage substantially eliminates, through laser or other high-energy radiation emissive source targeting and annihilating, the F/LT Ab-TAs complexes from the body fluid (blood plasma and/or blood). In an embodiment, the laser, or other high-energy radiation emissive source, may not have a beam in excess of 10 nanometers in diameter. The illumination source may be computer directed and/or controlled in real time. The body fluid, from which the morbid obesity Target Antigens: IL-1 (Interleukin-1), IL-6 (Interleukin-6), TNF-alpha (Tumor Necrosis Factor-alpha), cholesterol, C-Reactive Protein (CRP), Alpha-2-HS-glycoprotein (Fetuin-A), Inhibitor of Differentiation 1 (ID1), protein-tyrosine phosphatase (PTP)1B, monocyte chemoattractant protein-1 (MCP-1), IL-8 (Interleukin-8), haptoglobin, α-1 antitrypsin, α-1 acid-glycoprotein, and/or orosomucoid have been eradicated, may be returned to the patient, free of the morbid obesity target antigen(s). Persons having ordinary skill in art will recognize that the steps described above can be performed on various devices, machines, or systems. This disclosure contemplates all known devices, machines, or systems that can perform the steps described in the above illustrative example.

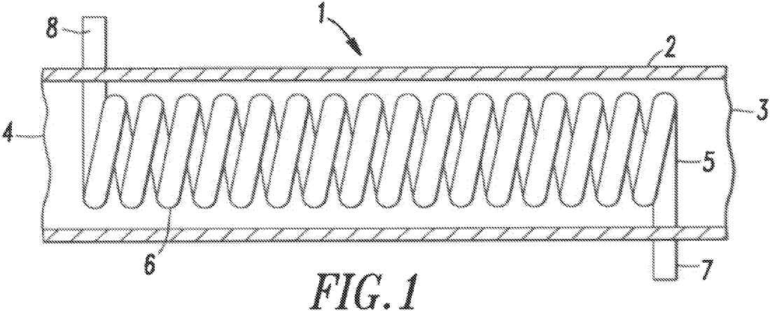

Referring to FIG. 1, in an embodiment, using a device, the F/LT Abs, targeting the targeted pathogenic antigen (TA: morbid obesity targeted pathogenic antigens) may be delivered in a concurrent or counter-current mode with reference to the flow of the body fluid. An example of a device that can be utilized in the disclosed method is shown in FIG. 1. The device 1 can include an exterior wall 2 to surround a treatment chamber 5. The treatment of the body fluid conveniently can be applied in the treatment chamber 5. Residence times of the body fluid in the device can be altered by changing the dimensions of the treatment chamber, or by using a dialysis vacuum pump. With reference to FIG. 1, body fluid enters the inlet 3, passes through the treatment chamber 5, and exits the outlet 4. The treatment of an antibody with an attached fluorescently tagged moiety or luminously tagged moiety (F/LT Ab) targeting the TPA can be applied from a delivery tube 6 located within the treatment chamber 5. In an embodiment, the F/LT Abs, targeting the morbid obesity TA(s) can be delivered in a concurrent or counter-current mode with reference to the flow of the body fluid. In counter-current mode, the body fluid enters the treatment chamber 5 at the inlet 3. The F/LT Ab-TAs can enter through a first lead 8 near the outlet 4 of the treatment chamber 5. Body fluid then passes to the outlet 4 and the F/LT Ab-TAs pass to the second lead 7 near the inlet 3.

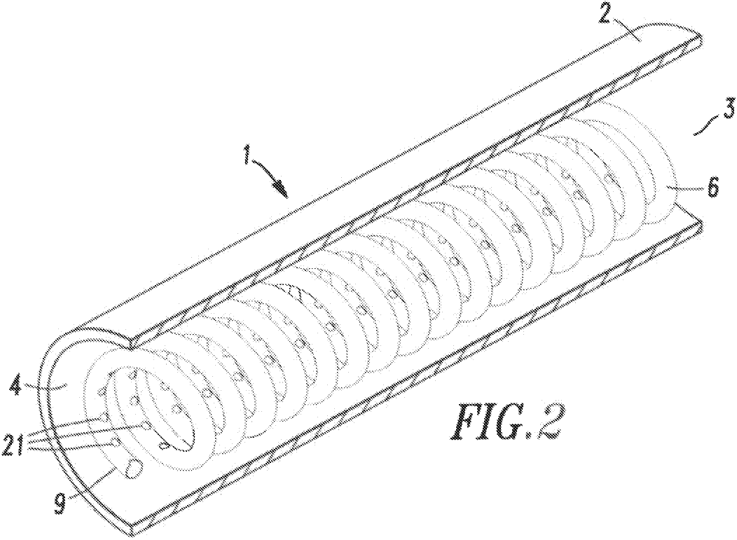

Referring to FIG. 2, in an embodiment, the delivery tube 6 can be hollow and with a plurality of holes 21. The F/LT Abs can be pumped through the delivery tube 6 to achieve a desired concentration of F/LT Abs in the body fluid. The F/LT Abs perfuse through the holes 21. An inferior wall 9 defines the delivery tube 6. The delivery tube 6 can include at least one lead 7, 8. The lead 7, 8 can deliver the treatment to the treatment chamber 5. Conveniently, the delivery tubes 6 will have a high contact surface area with the body fluid. As shown, the delivery tube 6 comprises a helical coil. The delivery tube 6 can include any suitable material including, for example, metal, plastic, ceramic, or combinations thereof. The delivery tube 6 can also be rigid or flexible. In one embodiment, the delivery tube 6 is a metal tube perforated with a plurality of holes. Alternatively, the delivery tube 6 can be plastic. FIG. 1 and FIG. 2 are non-limiting depictions of a device that can be used in the described method and are used for illustrative purposes only.

In an embodiment, as an alternative to using a type of device as described above, the morbid obesity Target Antigens may be captured using antibody microarrays containing fluorescent (Fl) or luminescent (Lu) antibodies (Fl-Ab/Lu-Ab) in microarrays. An antibody microarray may be a protein microarray; a collection of capture antibodies is fixed on a solid surface, such as glass, plastic and silicon chip for the purpose of detecting antigens. Antibody microarrays are composed of millions of identical monoclonal antibodies attached at high density on glass or plastic slides, all of which are transparent. Any microarrays known by those skilled in the art sufficient to perform the described technique are contemplated by this disclosure.

During the extracorporeal exposure of the TAs, there is created an antibody pathogen complex of FI-Ab TA or Lu-Ab-TA on the microarray. The complexes are then tracked using an appropriate sensor and obliterated using a high energy focused radiation beam such as a laser, which is less than 10 nanometer in diameter. All steps in the process may be monitored and controlled by a computer in real time. Persons having ordinary skill in art will recognize that the steps described above can be performed on various devices, machines, or systems. This disclosure contemplates all known devices, machines, or systems that can perform the steps described in the above illustrative example.

To eradicate the morbid obesity antibody complexes after exposure in the microarrays, the body fluid may be forced through a container constructed from a transparent material such as glass, or other material, which exposes the F/LT Ab-TAs to a light-sensing device. The sensing device also creates an enlarged, magnified visual image of the F/LT Ab-TAs. A concentrated and focused intense energy beam, such as light, is then used to properly illuminate the F/LT Ab-TAs within the body fluid, such as blood plasma and/or blood. Each F/LT Ab-TA is very rapidly identified and precisely located. The targeted F/LT Ab-TAs are identified and tracked using optical or digital enhancement or magnification. The very rapid (0.0001 to 0.1 microsecond, as a non-limiting example) location and tracking of each targeted F/LT Ab-TA is achieved using computer graphics and computer programs known in the art. An alternative methodology may use optical pattern recognition of the F/LT Ab-TAs. A very narrow beam laser or other high-energy radiation emissive source may then be used to annihilate the targeted F/LT Ab-TAs in the body fluid (blood plasma and/or blood). The radiation source uses very short bursts of less than a millisecond to annihilate the F/LT Ab-TAs. Persons having ordinary skill in art will recognize that the steps described above can be performed on various devices/machines. This disclosure contemplates all known devices, machines, or systems that can perform the steps described in the above illustrative example.

The temperature of the treated body fluid may be maintained at or around 98.6° F. via continuous cooling of the body fluid using a standard cooling apparatus. A constant thermostatic measurement and control system continuously monitors the process to maintain the body fluid temperature at 98.6° F. Persons having ordinary skill in art will recognize that the steps described above can be performed on various devices, machines, or systems. This disclosure contemplates all known devices, machines, or systems that can perform the steps described in the above illustrative example.

In an embodiment, the morbid obesity target antigen(s) /TA(s) may comprise: IL-1 (Interleukin-1), IL-6 (Interleukin-6), TNF-alpha (Tumor Necrosis Factor-alpha), cholesterol, C-Reactive Protein (CRP), Alpha-2-HS-glycoprotein (Fetuin-A), Inhibitor of Differentiation 1 (ID1), protein-tyrosine phosphatase (PTP)1B, monocyte chemoattractant protein-1 (MCP-1), IL-8 (Interleukin-8), haptoglobin, α-1 antitrypsin, α-1 acid-glycoprotein, and/or orosomucoid can be identified and differentiated using standard ELISA methodology. Identification can be done before treatment to determine which TAs are present in patient’s blood and after treatment to analyze the efficiency of removal of the TA. ELISA (enzyme-linked immunosorbant assay) is a biochemical technique which may allow for the detection of an antigen in a sample. In ELISA, an antigen may be affixed to a surface, and then an antibody is utilized for binding to the antigen. The antibody is linked to an enzyme which enables a color change in the substrate. Other strategies may be employed to validate the level of morbid obesity target antigen(s)/TA(s) in the body fluid before or after treatment: Western blotting technology, UV/V is spectroscopy, mass spectrometry, and surface plasmon resonance (SPR). Another alternative methodology would utilize a molecular weight cut-off filtration. Molecular weight cut-off filtration refers to the molecular weight at which at least 80% of the target antigen(s)/TA(s) is prohibited from membrane diffusion.

In an embodiment, a portion of the purified body fluid may be tested to ensure that an acceptable portion of the morbid obesity targeted antigen(s)/TA(s) has been successfully removed from the body fluid using methods discussed throughout this application. Testing can determine the length of treatment and evaluate the efficacy of laser and/or radiofrequency eradication methodology in removing the targeted antigens. Body fluid with an unacceptably high concentration of morbid obesity antibody complexes remaining can then be re-treated before returning the body fluid to the patient.

In an embodiment, the treatment may eradicate the targeted morbid obesity targeted antigen(s) and subsequently the targeted antigens from the body fluid. The cleansed body fluid can then be returned to the patient, for example by using the same catheter that was originally used in removing the body fluid. In one non-limiting embodiment, the treatment of blood and/or blood plasma comprises removing 20 ml to 40 ml of the body fluid from a patient, and then applying the treatment to the body fluid before returning it to the patient. The frequency of such treatments would depend upon an analysis of the underlying symptomatology and pathology of the patient.

In an embodiment, the antibody(s) may be fluorescent or luminous tagged antibody (F/LT Ab) generated to bind specific targeted pathogenic antigens (TPAs) of morbid obesity. In an embodiment, the antibody(s) may be an antibody with a metallic moiety generated to bind to specific targeted pathogenic antigens (TPAs) of morbid obesity. These antibodies will be susceptible to eradication through a laser treatment and/or radiofrequency treatment.

In an embodiment, the device may include an additional stage such as a removal module. In embodiments, the removal module can include, for example, a mechanical filter, a chemical filter, a dialysis machine, a magnet, a molecular filter, molecular adsorbant recirculating system (MARS), a plasmapharesis unit, and combinations thereof. In an embodiment, in addition to the extra-corporeal methodology for the removal of disease specific target antigen(s)/TAs, an embodiment could physically remove the antibody-metallic or moiety-target antigen from the body fluid. This may occur after the eradication and/or neutralization of the target antigen(s) by a radiofrequency (RF) or a laser technique. Alternatively, this could occur even without first utilizing any RF or laser technique against the target antigen, as the target antigen will have been physically separated from the patient’s body fluid prior to being reintroduced into the patient.

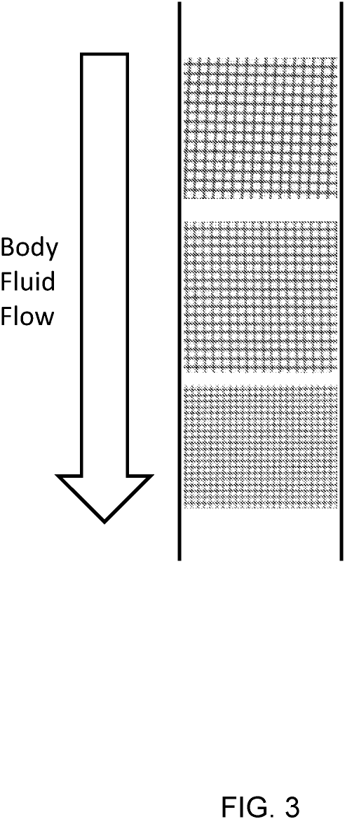

A methodology of the present intervention, which would separate the TA (target antigen(s) from re-entering the body fluid (blood plasma or blood) of the patient being treated may use a designer antibody with an attached macromolecular moiety, such as a metallic moiety. The macromolecular moiety, attached to the antibody, would be 1.000 mm to 0.0000001 mm in diameter. The antibody-macromolecular moiety-targeted antigen complex would then be blocked from reentering the patient’s blood, using a series of microscreens which contain openings with a diameter 50% to 99.99999% less than the diameter of the designer antibody-macromolecular moiety (See FIG. 3). The microscreen opening(s) may need to have a diameter of at least 25 µm (micrometer) to permit the passage and return to the circulation of the non-pathological blood constituents. However, the screen opening(s) may have much greater apertures when specifically dealing with CSF or blood plasma not containing blood elements such as white blood cells, red blood cells, platelets, etc. utilizing plasmapheresis techniques.

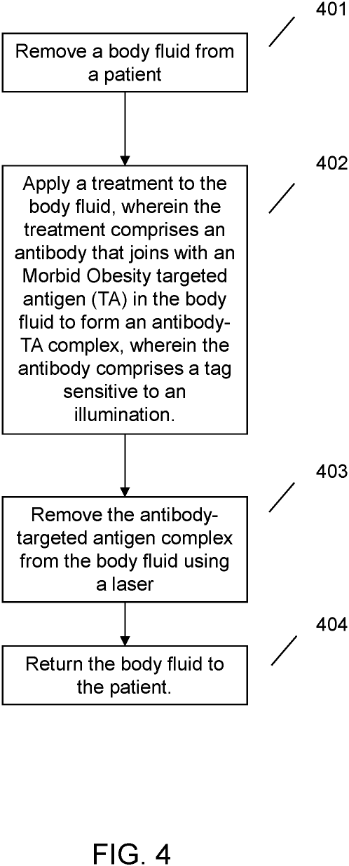

Referring to FIG. 4, an example method is illustrated. A method for treating a body fluid comprising: a first stage including removing the body fluid from a patient at 401; a second stage including applying a treatment to the body fluid wherein the treatment comprises an antibody that joins with a morbid obesity targeted antigen (TA) in the body fluid to form an antibody-TA complex, wherein the antibody comprises a tag sensitive to an illumination at 402, and removing the antibody-antigen complex from the body fluid with a laser at 403; and a third stage including returning the body fluid to the patient at 404.

Unless otherwise indicated, all numbers expressing quantities of ingredients, properties such as molecular weight, reaction conditions, and so forth used in the specification and claims are to be understood as being modified in all instances by the term “about.”

Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

While the invention has been particularly shown and described with reference to a preferred embodiment, it will be understood by those skilled in the art that various changes in form and detail may be made therein without departing from the spirit and scope of the invention.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data.

Embodiments may be implemented as an instrument, system, method or program product. Accordingly, an embodiment may take the form of an entirely hardware embodiment, or an embodiment including software (including firmware, resident software, micro-code, etc.) that may all generally be referred to herein as a “circuit,” “module” or “system.” Furthermore, embodiments may take the form of a program product embodied in at least one device readable medium having device readable program code embodied thereon.

A combination of device readable storage medium(s) may be utilized. In the context of this document, a device readable storage medium (“storage medium”) may be any tangible, non-signal medium that can contain or store a program comprised of program code configured for use by or in connection with an instruction execution system, apparatus, or device. For the purpose of this disclosure, a storage medium or device is to be construed as non-transitory, i.e., not inclusive of signals or propagating media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a hand-held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for treating a body fluid of a patient, comprising:
removing the body fluid from a patient;
applying a treatment to the body fluid, wherein the treatment comprises an antibody that joins with a morbid obesity targeted antigen (TA) in the body fluid to form an antibody-TA complex, wherein the antibody comprises a tag sensitive to an illumination;
removing the antibody-TA complex from the body fluid using an illumination source; and
returning the body fluid to the patient.

2. The method of claim 1, wherein the morbid obesity targeted antigen is selected from the group consisting of: IL-1 (Interleukin-1), IL-6 (Interleukin-6), TNF-alpha (Tumor Necrosis Factor-alpha), cholesterol, C-Reactive Protein (CRP), Alpha-2-HS-glycoprotein (Fetuin-A), Inhibitor of Differentiation 1 (ID1), protein-tyrosine phosphatase (PTP)1B, monocyte chemoattractant protein-1 (MCP-1), IL-8 (Interleukin-8), haptoglobin, α-1 antitrypsin, α-1 acid-glycoprotein, and orosomucoid.

3. The method of claim 1, wherein the tag is selected from the group consisting of: a fluorescent tag and a luminous tag.

4. The method of claim 1, wherein illumination source comprises a narrow beam laser with a beam less than 10 nanometers in diameter.

5. The method of claim 1, wherein the illumination source annihilates the antibody-target antigen (TA) complex and a disease-causing potential of the target antigen.

6. The method of claim 1, wherein energy of an emitted radiation of the illumination source annihilates the antibody-target antigen (TA) complex.

7. The method of claim 1, further comprising capturing the antibody-target antigen (TA) complex using an antibody microarray.

8. The method of claim 1, further comprising determining an efficacy of treatment, using the illumination source, based on testing the body fluid after the treating the body fluid and before returning the body fluid to the patient.

9. The method of claim 1, wherein the removal further comprises a sensor for detecting individual antibody-target antigen complexes.

10. The method of claim 1, wherein the body fluid is selected from the group consisting of: blood plasma and blood.

11. A method for treating a body fluid of a patient, comprising:
removing the body fluid from a patient;
applying a treatment to the body fluid, wherein the treatment comprises an antibody that joins with a morbid obesity targeted antigen (TA) in the body fluid to form an antibody-TA complex, wherein the antibody comprises a tag sensitive to illumination and a metallic moiety sensitive to a radiofrequency;
removing the antibody-TA complex from the body fluid using an illumination source; and returning the body fluid to the patient.

12. The method of claim 11, wherein the morbid obesity targeted antigen is selected from the group consisting of: IL-1 (Interleukin-1), IL-6 (Interleukin-6), TNF-alpha (Tumor Necrosis Factor-alpha), cholesterol, C-Reactive Protein (CRP), Alpha-2-HS-glycoprotein (Fetuin-A), Inhibitor of Differentiation 1 (ID1), protein-tyrosine phosphatase (PTP)1B, monocyte chemoattractant protein-1 (MCP-1), IL-8 (Interleukin-8), haptoglobin, α-1 antitrypsin, α-1 acid-glycoprotein, and/or orosomucoid.

13. The method of claim 11, wherein the tag is selected from the group consisting of: a fluorescent tag and a luminous tag.

14. The method of claim 11, wherein illumination source comprises a narrow beam laser with a beam less than 10 nanometers in diameter.

15. The method of claim 11, wherein the illumination source annihilates the antibody-target antigen (TA) complex and a disease-causing potential of the target antigen.

16. The method of claim 11, wherein energy of an emitted radiation of the illumination source annihilates the antibody-target antigen (TA) complex.

17. The method of claim 11, further comprising capturing the antibody-target antigen (TA) complex using an antibody microarray.

18. The method of claim 11, further comprising determining an efficacy of treatment, using the illumination source, based on testing the body fluid after the treating the body fluid and before returning the body fluid to the patient.

19. The method of claim 11, wherein the removal further comprises a sensor for detecting individual antibody-target antigen complexes.

20. A device for treating a body fluid of a patient, comprising:
a transparent first stage including an inlet for the body fluid and at least one exterior wall defining a treatment chamber;
a transparent second stage, fluidly connected to the first stage, comprising a removal module and an outlet for the body fluid, wherein the treatment chamber comprises a delivery tube for introducing an antibody into the treatment chamber, wherein the delivery tube comprises a hollow tube including at least one interior wall defining a plurality of holes through which the antibody can be added to the treatment chamber, wherein the treatment is delivered through the hollow tube in counter-current mode with reference to the body fluid, wherein the treatment comprises an antibody that joins with an morbid obesity targeted antigen (TA) in the body fluid to form an antibody-TA complex, wherein the antibody comprises a tag sensitive to an illumination; and
a laser illumination source.
